(19) United States
(12) Patent Application Publication
(10) Pub. No.: US 2005/0137177 A1
(43) Pub. Date: Jun. 23, 2005

(54) Ester combination local anesthetic

(76) Inventor: Steven L. Shafer, Mountain View, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 10/740,716

(22) Filed: Dec. 18, 2003

(51) Int. Cl. A61K 31/573; A61K 31/24; A61K 31/137

(52) U.S. Cl. 514/171; 514/537; 514/649

(57) ABSTRACT

The present invention provides compositions and methods for improved local anesthesia and/or analgesia, in which onset of action is rapid, the risk of toxicity is low, and the effect is sustained. More particularly, the present invention provides a combination of at least two ester anesthetics for administration to a subject, where at least one ester anesthetic provides a rapid onset of action and at least one ester anesthetic provides sustained activity. The compositions of the present invention are useful for the production of analgesia and/or anesthesia and are particularly useful for the prophylaxis and/or treatment of pain.

### CROSS-REFERENCES TO RELATED APPLICATIONS

The present application claims priority to U.S. Provisional Patent Application No., ______, filed Dec. 20, 2002, originally filed as U.S. patent application Ser. No. 10/327,310 the disclosure of which is hereby incorporated by reference in its entirety for all purposes.

### BACKGROUND OF THE INVENTION

Local anesthetics are drugs that produce reversible loss of sensation in a specific area of the body. Most of the clinically useful local anesthetic agents consist of an aromatic ring linked by a carbonyl containing moiety through a carbon chain to a substituted amino group. There are 2 major classes, defined by the nature of the carbonyl-containing linkage group. Agents with an ester linkage include cocaine, procaine, tetracaine, benzocaine, amethocaine and chloroprocaine. Those with an amide linkage include lidocaine, prilocaine, mepivacaine, ropivocaine, etidocaine, levobupivacaine and bupivacaine. There are important practical differences between these two classes of local anesthetic agents. Esters are chemically less stable than amides, and are rapidly hydrolyzed in the body by plasma cholinesterases and other esterases. This metabolism generally prevents esters from accumulating to toxic levels in vivo, even with repeated or prolonged doses. The amides are metabolized less rapidly, mainly by hepatic proteases, so they can build up to toxic levels with large or repeated doses. In current clinical practice, ester-based local anesthetics have largely been superseded by those in the amide class. Different amide-based local anesthetics are usually inactivated by a common pathway. More importantly, all local anesthetics share the same basic toxicity profile, e.g., seizures from central nervous system toxicity and arrhythmia and death from cardiac toxicity. The toxicities of all local anesthetics are additive. However, because amide-based local anesthetics have far slower metabolism than ester-based anesthetics, combinations of amide-based local anesthetics have the potential for additive toxicity from two compounds that are slowly eliminated by the body. The far more rapid metabolism of ester-based local anesthetics virtually precludes additive toxicity from drug absorption, although additive toxicity would be expected if two ester-based local anesthetics were given by direct intravenous injection. Similar problems are encountered with repeated doses of amide-based local anesthetics, where the toxicity of the second dose adds to that of the first dose, due to their slow metabolism and inactivation.

Local anesthetics interrupt the conduction of impulses in peripheral nerves by blocking sodium channels from the intracellular side of the cell membrane. This causes a local decrease in the rate and degree of depolarization of the nerve membrane, such that the threshold potential for transmission is not reached. There is no effect on the resting or threshold potential, although the refractory period and repolarization may be prolonged.

Topically, local anesthetics are applied to the skin, the eye, the ear, the nose, and the mouth, as well as other mucous membranes. Infiltration techniques are typically used to provide anesthesia for minor surgical procedures. Amide anesthetics with a moderate duration of action are commonly used. The site of action is at unmyelinated nerve endings. Onset is very rapid with infiltration techniques. However, the duration of local anesthesia is variable, and depends on the amount of drug injected as well as the physical properties of the local anesthetic. In general, local anesthetics that are more soluble in lipid have a slower onset, but a longer duration of effect, than local anesthetics that are less soluble in lipid. Conduction anesthesia can be divided into minor nerve blockade and major blockade of deeper nerves or trunks with a wide dermatomal distribution. Onset of conduction anesthesia ranges from several minutes for local anesthetics that are relatively insoluble in lipid, to nearly an hour for local anesthetics that are highly soluble in lipid. Duration of conduction anesthesia varies from 60 minutes for local anesthetics that are relatively insoluble in lipid to many hours for local anesthetics that are highly soluble in lipid. Local anesthetic solutions can also be deposited in the epidural space. The injected local anesthetic solution produces analgesia by blocking conduction at the intradural spinal nerve roots. Again, the lipid soluble local anesthetics will have slower onset, but longer duration of effect, than the relatively lipid insoluble local anesthetics.

Systemic and localized toxic reactions may occur, e.g., from the accidental intravascular or intrathecal injection, of an excessive administration dose, etc. Systemic reactions to local anesthetics involve primarily the central nervous system (CNS) and the cardiovascular system. The initial symptoms of CNS toxicity involve feelings of light-headedness, dizziness and circumoral paraesthesia, which may precede visual and/or auditory disturbances such as difficulty focusing and tinnitus. Other signs include shivering, muscular twitching, and tremors initially involving muscles of the face and distal parts of the extremities. Ultimately, generalized convulsions occur, progressing to CNS depression and coma. Respiratory depression may result in respiratory arrest.

Cardiovascular toxicity usually occurs at doses and blood concentrations that are higher than those required to produce CNS toxicity. Extremely high concentrations of local anesthetics depress spontaneous pacemaker activity in the sinus node resulting in sinus bradycardia and sinus arrest. They also exert a negative ionotropic action on isolated cardiac tissue. Bupivacaine and etidocaine have been reported to cause rapid and profound cardiovascular depression in some patients following accidental intravascular injection. In fact, a review of the Medline database reveals that 102 deaths from bupivacaine (e.g., heart arrest, myocardial infarction, accidental overdose, etc.) occurred between 1990 and 1997.

Currently used local anesthetics often suffer from a limited duration of action, which is too short to relieve most postoperative pain, or from a slow onset of effect, which limits utility in the operating room where rapid onset is necessary to avoid delaying surgery. The onset of activity is a particularly important issue for local anesthesia, in contrast to spinal anesthesia where the onset of anesthetic effect is invariably rapid. See, for example, Hauch et al., Reg. Anesth. 15:81-85 (1990). Amide local anesthetics often suffer from safety concerns, as systemic and localized toxic reactions to these anesthetics lead to CNS and cardiovascular toxicity. In addition, currently used local anesthetics such as lidocaine produce pain and discomfort (e.g., stinging) when administered.

In view of the foregoing, there has been a long-standing need in the art for local anesthetic and/or analgesic formulations that have a rapid onset, a long duration of action, minimal toxic side-effects, and ease of administration. The present invention satisfies this and other needs by providing improved long-lasting local anesthetic and/or analgesic formulations that produce a more prolonged duration of nerve blockade, faster onset of action, minimal toxicity, greater efficacy, and ease of administration.

### BRIEF SUMMARY OF THE INVENTION

The present invention provides compositions and methods for improved local anesthesia and/or analgesia, in which the onset of action is rapid, the risk of toxicity is low, and the effect is sustained. At least two ester anesthetics are combined, where at least one provides a rapid onset of action and at least one provides sustained activity. Importantly, because the combination of ester anesthetics produces minimal toxicity due to very rapid metabolism in the blood, each can be given in quantities sufficient to exert its full pharmacological benefit. For example, both the rapid onset ester anesthetic and the long-lasting ester anesthetic are administered at full dose, providing a rapid onset of effect and a full duration of effect, respectively. The low risk of toxicity associated with administering a combination of ester anesthetics also provides the possibility for repeated administrations of a full dose, which cannot be done safely with amide anesthetics. The compositions are used for the production of analgesia and/or anesthesia and are particularly useful for the prophylaxis and/or treatment of pain. As local anesthetics, the compositions are useful for regional anesthesia, e.g., topical anesthesia, infiltration anesthesia, field block anesthesia, peripheral nerve block anesthesia, epidural anesthesia, spinal anesthesia, bier block anesthesia, and combinations thereof.

In one aspect, the present invention provides a method for local anesthesia, the method comprising administering a combination of at least two ester anesthetics.

In another aspect, the present invention provides a method for analgesia, the method comprising administering a combination of at least two ester anesthetics.

In yet another aspect, the present invention provides a pharmaceutical composition for local anesthesia, the pharmaceutical composition comprising:
(a) a combination of at least two ester anesthetics; and
(b) a pharmaceutically acceptable carrier.

In still yet another aspect, the present invention provides a pharmaceutical composition for analgesia, the pharmaceutical composition comprising:
(a) a combination of at least two ester anesthetics; and
(b) a pharmaceutically acceptable carrier.

### BRIEF DESCRIPTION OF THE DRAWINGS

FIG. 1 shows the time course of sciatic nerve block with 2.0% lidocaine (upper panel), 0.5% bupivacaine (middle panel), and a combination of 0.17% tetracaine and 2.3% 2-chloroprocaine (lower panel).

FIG. 2 shows the onset and duration of a rat sciatic nerve block after administration of 2% 2-chloroprocaine.

FIG. 3 shows the onset and duration of a sciatic nerve block after administration of either 0.22% tetracaine (upper panel) or 0.5% tetracaine (lower panel).

### DETAILED DESCRIPTION OF THE INVENTION

I. Definitions

As used herein, the following terms have the meanings ascribed to them unless specified otherwise.

As used herein, the singular forms “a,” “an,” and “the” include plural references unless the context clearly dictates otherwise. Unless defined otherwise, all technical and scientific terms used herein have the same meaning as commonly understood to one of ordinary skill in the art to which the present invention belongs. Although any methods, devices, and materials similar or equivalent to those described herein can be used in the practice or testing of the invention, the preferred methods, devices, and materials are described herein.

The term “local anesthesia” refers to anesthesia characterized by the loss of sensation only in the area of the body where an anesthetic agent or a combination of anesthetic agents is administered. Local anesthesia can result, for example, from contact of an anesthetically effective amount of a local anesthetic with sensory nerve processes at the site at which the painful stimulus is present, or can result from inhibition of nerve transmission at a nerve or nerves proximal to the site at which the painful stimulus is present. As used herein, the term “anesthetically effective amount” refers to an amount of an anesthetic agent or a combination of anesthetic agents that produces an anesthetic effect, e.g., a partial or total loss of sensation, inhibition of sensory perception, or inhibition of motor function. Preferably, the anesthetically effective amount produces minimal toxic side-effects.

The term “anesthetic” refers to an agent that causes loss of sensation in a human or other mammal with or without the loss of consciousness. More particularly, the term “local anesthetic” refers to an anesthetic agent that induces local anesthesia by reversibly inhibiting peripheral nerve excitation and/or conduction. Local anesthetics suitable for use in the present invention include, but are not limited to, ester-based anesthetics, amide-based anesthetics, ester analogs of amide-based anesthetics, and ester analogs of other anesthetics. Ester-based anesthetics include, but are not limited to, cocaine, procaine, 2-chloroprocaine, tetracaine, benzocaine, amethocaine, chlorocaine, butamben, dibucaine, and the like. Amide-based anesthetics include, but are not limited to, lidocaine, prilocaine, mepivacaine, ropivocaine, etidocaine, levobupivacaine, bupivacaine, and the like. Other anesthetics suitable for use in the present invention include, but are not limited to, ester analogs of aconitine, dyclonine, ketamine, pramoxine, safrole, and salicyl alcohol. Such ester analogs can contain an ester group anywhere within the structure.

The terms “ester anesthetic” and “ester-based anesthetic” are used interchangeably herein to refer to the class of compounds having the structure set forth in Formula 1: 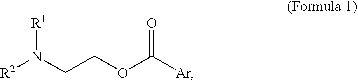
wherein R¹ and R² are independently selected from the group consisting of H and a structural fragment having a saturated or unsaturated linear, branched, or cyclic skeleton containing one to ten carbon atoms in which the carbon atoms may be optionally substituted with a substituent selected from the group consisting of —OH, —OR³, —O₂CR³, —SH, —SR³, —SOCR³, —NH₂, —NHR³, NH(R³)₂, —NHCOR³, —NRCOR³, —I, —Br, —Cl, —F, —CN, —CO₂H, —CO₂R³, —CHO, —COR³, —CONH₂, —CONHR³, —CON(R³)₂, —COSH, —COSR³, —NO₂, —SO₃H, —SOR³, and —SO₂R³, wherein R³ is a saturated or unsaturated linear, branched or cyclic alkyl group containing one to ten carbons, and wherein Ar is an aromatic substituent selected from the group consisting of phenyl, naphthyl, anthracyl, phenanthryl, furan, pyrrole, thiophene, benzofuran, benzothiophene, quinoline, isoquinoline, imidazole, thiazole, oxazole, and pyridine. Ar may be optionally substituted with a substituent selected from the group consisting of —OH, —OR, —O₂CR, —SH, —SR, —SOCR, —NH₂, —NHR, —NHCl, —NH(R)₂, —NHCOR, —NRCOR, —I, —Br, —Cl, —F, —CN—, —CO₂H, —CO₂R, —CHO, —COR, —CONH₂, —CONHR, —CON(R)₂, —COSH, —COSR, —NO₂, —SO₃H, —SOR, and —SO₂R, wherein R is the same as defined above for R¹ and R². Other ester anesthetics including, but not limited to, benzoylecgonine, butacaine, cocaethylene, and meperidine are also within the scope of the present invention.

The term “ester analogs of amide-based anesthetics” refers to amide-based anesthetic compounds wherein the amide (N—H) group is replaced with an oxygen (O) atom, as set forth in Formula 2: 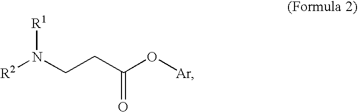
wherein R¹ and R² are independently selected from the group consisting of H and a structural fragment having a saturated or unsaturated linear, branched, or cyclic skeleton containing one to ten carbon atoms in which the carbon atoms may be optionally substituted with a substituent selected from the group consisting of —OH, —OR³, —O₂CR³, —SH, —SR³, —SOCR³, —NH₂, —NHR³, NH(R³)₂, —NHCOR³, —NRCOR³, —I, —Br, —Cl, —F, —CN, —CO₂H, —CO₂R³, —CHO, —COR³, —CONH₂, —CONHR³, —CON(R³)₂, —COSH, —COSR³, —NO₂, —SO₃H, —SOR³, and —SO₂R³, wherein R³ is a saturated or unsaturated linear, branched or cyclic alkyl group containing one to ten carbons, and wherein Ar is an aromatic substituent selected from the group consisting of phenyl, naphthyl, anthracyl, phenanthryl, furan, pyrrole, thiophene, benzofuran, benzothiophene, quinoline, isoquinoline, imidazole, thiazole, oxazole, and pyridine. Ar may be optionally substituted with a substituent selected from the group consisting of —OH, —OR, —O₂CR, —SH, —SR, —SOCR, —NH₂, —NHR, —NHCl, —NH(R)₂, —NHCOR, —NRCOR, —I, —Br, —Cl, —F, —CN—, —CO₂H, —CO₂R, —CHO, —COR, —CONH₂, —CONHR, —CON(R)₂, —COSH, —COSR, —NO₂, —SO₃H, —SOR, and —SO₂R, wherein R is the same as defined above for R¹ and R². Examples of ester analogs of amide-based anesthetics include, but are not limited to, ester analogs of lidocaine, prilocaine, mepivacaine, ropivocaine, etidocaine, levobupivacaine, bupivacaine, dibucaine, and the like. Other ester analogs of amide-based anesthetics including, but not limited to, oxethazine, pentobarbital, thiamylal, and thiopental are also within the scope of the present invention. Such ester analogs have an amide (N—H) group that is replaced with an oxygen (O) atom.

The term “analgesia” refers generally to the reduction of pain or to the full elimination of pain in a human or other mammal suffering from pain. As used herein, the term “analgesically effective amount” refers to an amount of an anesthetic agent or a combination of anesthetic agents that produces a reduction of pain or a full elimination of pain in a human or other mammal. Preferably, the analgesically effective amount produces minimal toxic side-effects.

The term “unit dosage forms” refers to physically discrete units suitable as unitary dosages for human subjects and other mammals. Each unit contains a predetermined quantity of active material calculated to produce the desired onset, tolerability, and therapeutic effects, in association with a suitable pharmaceutical excipient (e.g., an ampoule).

The term “administering” refers to oral administration, administration as a suppository, topical contact, mucosal administration, or administration by parenteral routes, e.g., intradermal, intravenous, subcutaneous, intramuscular, intra-arteriole, intraperitoneal, intraventricular, intracranial, epidural, spinal, rectal, vaginal, and the like, to a subject. Other modes of delivery include, but are not limited to, the use of liposomal formulations, intravenous infusion, the implantation of a slow-release device (e.g., a mini-osmotic pump), transdermal patches, etc. The most suitable route will depend on the nature and severity of the condition being treated. Infiltration methods (e.g., injection methods), which include subcutaneous injection, injection into perisurgical tissue, peripheral nerve blocks, bier block intravenous regional anesthesia, serosal, and neuraxial delivery (e.g., epidural, caudal, etc.), are preferred routes for the compounds of the present invention. Other suitable methods of administration include, but are not limited to, the placement of plegetts coated with a combination of ester anesthetics, intratracheal ester anesthetic administration, and ester anesthetic administration in the balloon of an endotracheal tube.

The term “combination of at least two ester anesthetics” refers to at least two ester anesthetic compounds delivered in either a simultaneous or sequential manner such that the combination provides improved local anesthesia with a rapid onset, a long duration of action, minimal toxicity, and/or ease of administration. Such combinations are safe enough to be administered repeatedly by any method known to one skilled in the art.

The term “vasoconstrictor” refers to an agent that induces or initiates vasoconstriction, thereby enhancing the potency, reducing the maximum systemic concentration, and prolonging the duration of action of an ester anesthetic of the present invention by localizing the active anesthetic in local tissues. Suitable vasoconstrictors include, but are not limited to, adrenaline (epinephrine), phenylepinephrine, angiotensin, phenylpropanolamine, and combinations thereof. Adrenaline (epinephrine) and phenylepinephrine are commonly used vasoconstrictors and may be added in concentrations ranging from about 1 in 80,000 to 1 in 500,000, preferably at a concentration of about 1 in 200,000 (5 μg/ml).

The term “corticosteroid” refers to any of the steroid hormones produced by the adrenal cortex or their synthetic equivalents, such as cortisol and aldosterone. Corticosteroids that are useful in the present invention to prolong in vivo nerve blockade include, but are not limited to, glucocorticoids such as dexamethasone, cortisone, hydrocortisone, prednisone, other glucocorticoids routinely administered orally or by injection, and pharmaceutically acceptable salts and combinations thereof. Other glucocorticoids include beclomethasone, betamethasone, flunisolide, methyl prednisone, paramethasone, prednisolone, triamcinolome, alclometasone, amcinonide, clobetasol, fludrocortisone, diflurosone diacetate, fluocinolone acetonide, fluoromethalone, flurandrenolide, halcinonide, medrysone, mometasone, and pharmaceutically acceptable salts and combinations thereof.

The term “permeability enhancer” refers to an agent the aids in the passage of an ester anesthetic into a tissue or across a cell membrane. Suitable permeability enhancers for use in the present invention include, but are not limited to, bile salts such as sodium cholate, sodium glycocholate, sodium glycodeoxycholate, taurodeoxycholate, sodium deoxycholate, sodium lithocholate chenocholate, chenodeoxycholate, ursocholate, ursodeoxycholate, hydrodeoxycholate, dehydrocholate, glycochenocholate, taurochenocholate, and taurochenodeoxycholate. Other permeation enhancers such as urea, sodium dodecyl sulfate (SDS), dimethyl sulfoxide (DMSO), sodium lauryl sulfate, salts and other derivatives of saturated and unsaturated fatty acids, surfactants, bile salt analogs, derivatives of bile salts, or such synthetic permeation enhancers as described in U.S. Pat. No. 4,746,508 may also be used.

The terms “lipophilic solvent” and “amphiphilic solvent” are used interchangeably herein to refer to solvents that can be added to the compositions of the present invention to prolong in vivo nerve blockade. Such solvents are well known to those skilled in the art and available from a variety of commercial sources. Examples of solvents suitable for use in the present invention include, but are not limited to, alcohols such as ethanol added in a dosage equivalent to approximately 1% alcohol, and polyoxyethylene sorbitan derivatives such as polysorbate-80 or Tween, added in a concentration equivalent to between about 1% and about 3%.

The term “buffering agent” refers to a substance that minimizes change in the acidity of a solution when an acid or base is added to the solution. Such buffering agents are well known to those skilled in the art. Typically, the compositions of the present invention are buffered, e.g., with bicarbonate, to maintain a mildly acidic or mildly alkaline pH. Buffering the solution also hastens the speed of onset and enhances the duration of drug effect. Generally, the compositions of the present invention are buffered to maintain the highest possible pH that is not associated with anesthetic precipitation and to provide an appropriate shelf-life. The pH is then adjusted to minimize acid- and/or base-catalyzed hydrolysis of the ester group of an ester anesthetic in vitro.

The term “rapid onset of action” refers to the onset of an anesthetic effect beginning within at least 10 minutes of anesthetic administration, e.g., when delivered for infiltration anesthesia, and may be at least about 5 minutes, preferably at least about 2 minutes, or less. Suitable anesthetics for this purpose include procaine and 2-chloroprocaine. Preferably, the anesthetic is 2-chloroprocaine.

The terms “sustained activity,” “extended period of action,” and “long duration of effect” are used interchangeably herein to refer to the situation where an anesthetic effect from a single dose, for example, when delivered by infiltration anesthesia, is maintained for at least about 1 hour, preferably at least about 2 hours, more preferably at least 4 hours, still more preferably at least about 6 hours, or more.

II. General Overview

The present invention provides compositions and methods for improved local anesthesia and/or analgesia characterized by a rapid onset of action, a long duration of effect, and a low risk of toxicity. At least two ester anesthetics are combined, where at least one provides a rapid onset of action and at least one provides sustained activity. Importantly, because the combination of ester anesthetics produces minimal toxicity due to very rapid metabolism in the blood, each can be given in quantities both safe and sufficient to provide a rapid onset and a long duration of action of local anesthesia. In contrast, combinations of anesthetically effective amounts of amide anesthetics are not possible due to the high risk of toxic side-effects. In addition, the low risk of toxicity associated with administering a combination of ester anesthetics provides the possibility for repeated administrations, which cannot be done safely with amide anesthetics. The compositions of the present invention are used for the production of analgesia and/or anesthesia and are particularly useful for the prophylaxis and/or treatment of pain. As local anesthetics, the compositions are useful for regional anesthesia, e.g., topical anesthesia, infiltration anesthesia, field block anesthesia, peripheral nerve block anesthesia, epidural anesthesia, spinal anesthesia, bier block anesthesia, and combinations thereof.

III. Description of the Embodiments

In one aspect, the present invention provides a method for local anesthesia, the method comprising administering a combination of at least two ester anesthetics.

In one embodiment, at least one of the ester anesthetics provides a rapid onset of action, and at least one of the ester anesthetics provides a long duration of effect. In another embodiment, the combination of at least two ester anesthetics produces minimal toxic side-effects. In yet another embodiment, the combination of at least two ester anesthetics is administered repeatedly, e.g., at least 2, 3, 4, 5, 6, 7, 8, or more times. In still yet another embodiment, the combination of at least two ester anesthetics is administered by continuous infusion. In a further embodiment, the combination of at least two ester anesthetics is administered either acutely or chronically for the prophylaxis and/or treatment of pain. One skilled in the art will know of the length of acute or chronic administration necessary to achieve such prophylaxis and/or treatment.

In a preferred embodiment, one of the ester anesthetics is procaine or 2-chloroprocaine. Preferably, the ester anesthetic is 2-chloroprocaine. In another preferred embodiment, one of the ester anesthetics is tetracaine. In a particularly preferred embodiment, one of the ester anesthetics is 2-chloroprocaine and one of the ester anesthetics is tetracaine. In one embodiment, the combination of 2-chloroprocaine and tetracaine is used for regional anesthesia selected from the group consisting of topical anesthesia, infiltration anesthesia, field block anesthesia, peripheral nerve block anesthesia, epidural anesthesia, spinal anesthesia, bier block anesthesia, and combinations thereof.

In a further embodiment, the combination of at least two ester anesthetics is administered as a single pharmaceutical formulation. In a first embodiment, the pharmaceutical formulation further comprises a buffering agent. In a second embodiment, the pharmaceutical formulation further comprises a vasoconstrictive agent. Preferably, the vasoconstrictive agent is epinephrine or phenylepinephrine. In a third embodiment, the pharmaceutical formulation further comprises a corticosteroid. Preferably, the corticosteroid is a glucocorticoid selected from the group consisting of dexamethasone, cortisone, hydrocortisone, prednisone, other glucocorticoids routinely administered orally or by injection, and pharmaceutically acceptable salts and combinations thereof. In a fourth embodiment, the pharmaceutical formulation further comprises a tissue permeability enhancer, as described above. In a fifth embodiment, the local anesthetic formulation is supplied as a liquid (e.g., solution). Preferably, the liquid contains the local anesthetics and adjuvants in suitable concentrations for immediate use without further mixing or dilution. In a sixth embodiment, the local anesthetic formulation is supplied as a lyophilized powder that can be reconstituted with water prior to injection.

In yet another embodiment, the combination of at least two ester anesthetics is delivered topically or by infiltration. Preferably, delivery by infiltration achieves a peripheral nerve block, an epidural nerve block, a caudal nerve block, or combinations thereof.

In another aspect, the present invention provides a method for analgesia, the method comprising administering a combination of at least two ester anesthetics.

In one embodiment, at least one of the ester anesthetics provides a rapid onset of action, and at least one of the ester anesthetics provides a long duration of effect. In another embodiment, the combination of at least two ester anesthetics produces minimal toxic side-effects. In yet another embodiment, the combination of at least two ester anesthetics is administered repeatedly, e.g., at least 2, 3, 4, 5, 6, 7, 8, or more times. In still yet another embodiment, the combination of at least two ester anesthetics is administered by continuous infusion. In a further embodiment, the combination of at least two ester anesthetics is administered either acutely or chronically for the prophylaxis and/or treatment of pain. One skilled in the art will know of the length of acute or chronic administration necessary to achieve such prophylaxis and/or treatment. In yet a further embodiment, an analgesically effective amount of the ester anesthetics provides prophylaxis and/or treatment for neuropathic pain, trauma, or tissue ischemia.

In a preferred embodiment, one of the ester anesthetics is procaine or 2-chloroprocaine. Preferably, the ester anesthetic is 2-chloroprocaine. In another preferred embodiment, one of the ester anesthetics is tetracaine. In a particularly preferred embodiment, one of the ester anesthetics is 2-chloroprocaine and one of the ester anesthetics is tetracaine. In one embodiment, the combination of 2-chloroprocaine and tetracaine is used for the prophylaxis and/or treatment of pain.

In a further embodiment, the combination of at least two ester anesthetics is administered as a single pharmaceutical formulation. In a first embodiment, the pharmaceutical formulation further comprises a buffering agent. In a second embodiment, the pharmaceutical formulation further comprises a vasoconstrictive agent. Preferably, the vasoconstrictive agent is epinephrine or phenylepinephrine. In a third embodiment, the pharmaceutical formulation further comprises a corticosteroid. Preferably, the corticosteroid is a glucocorticoid selected from the group consisting of dexamethasone, cortisone, hydrocortisone, prednisone, other glucocorticoids routinely administered orally or by injection, and pharmaceutically acceptable salts and combinations thereof. In a fourth embodiment, the pharmaceutical formulation further comprises a tissue permeability enhancer, as described above. In a fifth embodiment, the local anesthetic formulation is supplied as a liquid (e.g., solution). Preferably, the liquid contains the local anesthetics and adjuvants in suitable concentrations for immediate use without further mixing or dilution. In a sixth embodiment, the local anesthetic formulation is supplied as a lyophilized powder that can be reconstituted with water prior to injection.

In yet another embodiment, the combination of at least two ester anesthetics is delivered topically or by infiltration. Preferably, delivery by infiltration achieves a peripheral nerve block, an epidural nerve block, a caudal nerve block, or combinations thereof.

In yet another aspect, the present invention provides a pharmaceutical composition for local anesthesia, the pharmaceutical composition comprising:
(a) a combination of at least two ester anesthetics; and
(b) a pharmaceutically acceptable carrier.

In one embodiment, at least one of the ester anesthetics provides a rapid onset of action, and at least one of the ester anesthetics provides a long duration of effect. In another embodiment, the combination of at least two ester anesthetics produces minimal toxic side-effects. In yet another embodiment, the combination of at least two ester anesthetics is administered repeatedly, e.g., at least 2, 3, 4, 5, 6, 7, 8, or more times. In still yet another embodiment, the combination of at least two ester anesthetics is administered by continuous infusion. In a further embodiment, the combination of at least two ester anesthetics is administered either acutely or chronically for the prophylaxis and/or treatment of pain.

In a preferred embodiment, one of the ester anesthetics is procaine or 2-chloroprocaine. Preferably, the ester anesthetic is 2-chloroprocaine. In another preferred embodiment, one of the ester anesthetics is tetracaine. In a particularly preferred embodiment, one of the ester anesthetics is 2-chloroprocaine and one of the ester anesthetics is tetracaine. In one embodiment, 2-chloroprocaine is present at a concentration of from about 0.1% to about 10%, preferably from about 1% to about 6%, more preferably from about 1% to about 3%. In another embodiment, tetracaine is present at a concentration of from about 0.05% to about 1%, preferably from about 0.1% to about 0.75%, more preferably from about 0.1% to about 0.5%. In a further embodiment, the combination of 2-chloroprocaine and tetracaine is used for regional anesthesia selected from the group consisting of topical anesthesia, infiltration anesthesia, field block anesthesia, peripheral nerve block anesthesia, epidural anesthesia, spinal anesthesia, bier block anesthesia, and combinations thereof.

In another embodiment, the pharmaceutical formulation further comprises a buffering agent. In yet another embodiment, the pharmaceutical formulation further comprises a vasoconstrictive agent. Preferably, the vasoconstrictive agent is epinephrine or phenylepinephrine. In still yet another embodiment, the pharmaceutical formulation further comprises a corticosteroid. Preferably, the corticosteroid is a glucocorticoid selected from the group consisting of dexamethasone, cortisone, hydrocortisone, prednisone, other glucocorticoids routinely administered orally or by injection, and pharmaceutically acceptable salts and combinations thereof. In a further embodiment, the pharmaceutical formulation further comprises a tissue permeability enhancer, as described above. In yet a further embodiment, the local anesthetic formulation is supplied as a liquid (e.g., solution). Preferably, the liquid contains the local anesthetics and adjuvants in suitable concentrations for immediate use without further mixing or dilution. In still yet a further embodiment, the local anesthetic formulation is supplied as a lyophilized powder that can be reconstituted with water prior to injection.

In still yet another aspect, the present invention provides a pharmaceutical composition for analgesia, the pharmaceutical composition comprising:
(a) a combination of at least two ester anesthetics; and
(b) a pharmaceutically acceptable carrier.

In one embodiment, at least one of the ester anesthetics provides a rapid onset of action, and at least one of the ester anesthetics provides a long duration of effect. In another embodiment, the combination of at least two ester anesthetics produces minimal toxic side-effects. In yet another embodiment, the combination of at least two ester anesthetics is administered repeatedly, e.g., at least 2, 3, 4, 5, 6, 7, 8, or more times. In still yet another embodiment, the combination of at least two ester anesthetics is administered by continuous infusion. In a further embodiment, the combination of at least two ester anesthetics is administered either acutely or chronically for the prophylaxis and/or treatment of pain. In a further embodiment, an analgesically effective amount of the ester anesthetics provides prophylaxis and/or treatment for neuropathic pain, trauma, or tissue ischemia.

In a preferred embodiment, one of the ester anesthetics is procaine or 2-chloroprocaine. Preferably, the ester anesthetic is 2-chloroprocaine. In another preferred embodiment, one of the ester anesthetics is tetracaine. In a particularly preferred embodiment, one of the ester anesthetics is 2-chloroprocaine and one of the ester anesthetics is tetracaine. In one embodiment, 2-chloroprocaine is present at a concentration of from about 0.1% to about 10%, preferably from about 1% to about 6%, more preferably from about 1% to about 3%. In another embodiment, tetracaine is present at a concentration of from about 0.05% to about 1%, preferably from about 0.1% to about 0.75%, more preferably from about 0.1% to about 0.5%. In one embodiment, the combination of 2-chloroprocaine and tetracaine is used for the prophylaxis and/or treatment of pain.

In another embodiment, the pharmaceutical formulation further comprises a buffering agent. In yet another embodiment, the pharmaceutical formulation further comprises a vasoconstrictive agent. Preferably, the vasoconstrictive agent is epinephrine or phenylepinephrine. In still yet another embodiment, the pharmaceutical formulation further comprises a corticosteroid. Preferably, the corticosteroid is a glucocorticoid selected from the group consisting of dexamethasone, cortisone, hydrocortisone, prednisone, other glucocorticoids routinely administered orally or by injection, and pharmaceutically acceptable salts and combinations thereof. In a further embodiment, the pharmaceutical formulation further comprises a tissue permeability enhancer, as described above. In yet a further embodiment, the local anesthetic formulation is supplied as a liquid (e.g., solution). Preferably, the liquid contains the local anesthetics and adjuvants in suitable concentrations for immediate use without further mixing or dilution. In still yet a further embodiment, the local anesthetic formulation is supplied as a lyophilized powder that can be reconstituted with water prior to injection.

IV. Compositions

Most local anesthetics are bases that are almost insoluble in water unless protonated. Solubility is greatly increased by preparation of protonated amine salts of the amine moiety contained within the ester anesthetic. These salts are commonly prepared by the reaction of various inorganic and organic acids with the basic amine moiety of the anesthetic to produce a protonated amine salt. Representative inorganic acids may include hydrochloric, sulfuric, phosphoric, etc. Representative organic acids include acetic, benzoic, oxalic, citric, methylsulfonic, etc. The above notwithstanding, the local anesthetic may also be supplied as a water/lipid emulsion, in which at least one of the local anesthetics is delivered as the unprotonated basic amine moiety.

For injection use, e.g. infiltration methods, the combination of ester anesthetics will be provided as a dilute solution. For example, 2-chloroprocaine may be present at a concentration from about 0.1% to about 10%, preferably from about 1% to about 6%, more preferably from about 1% to about 3%, still more preferably about 1.0, 1.5, 2.0, 2.3, 2.5, or 3%, where a solution expressed as 1% contains 1 g of substance in 100 ml. Therefore, a 1% solution of 2-chloroprocaine contains 10 mg/ml of 2-chloroprocaine. Tetracaine may be present at a concentration from about 0.05% to about 1%, preferably from about 0.1% to about 0.75%, more preferably from about 0.1% to about 0.5%, still more preferably about 0.1, 0.17, 0.2, 0.22, 0.3, 0.4, or 0.5%. Typically, the solution will be buffered, e.g., with bicarbonate, to maintain a mildly acidic or mildly alkaline pH. Buffering the solution also hastens the speed of onset and enhances the duration of drug effect, but is limited by the tendency of local anesthetics to precipitate in solution under highly alkaline conditions. Generally, the solutions will be buffered to maintain the highest possible pH that is not associated with anesthetic precipitation and provides the appropriate shelf-life for the solution. The pH will be adjusted to minimize acid and/or base catalyzed hydrolysis of the ester group in vitro. In the case of a water/lipid emulsion that delivers at least one of the local anesthetics as an unprotonated basic amine moiety, the pH will be adjusted as necessary to maintain the local anesthetic in the unprotonated state.

The compositions of the present invention can also be provided in a lyophilized form. Such compositions may include a buffer, e.g., bicarbonate, for reconstitution prior to administration, or the buffer may be included in the lyophilized composition for reconstitution with, e.g., water. The lyophilized composition may further comprise a suitable vasoconstrictor, e.g., epinephrine. In one embodiment of the present invention, the lyophilized composition is provided in a syringe, optionally packaged in combination with the buffer for reconstitution, such that the reconstituted anesthetic composition can be immediately administered to a patient.

Pharmaceutical compositions will contain, as the active ingredient, the combination of ester anesthetics or a pharmaceutically acceptable salt thereof and one or more pharmaceutically acceptable excipients, carriers, diluents, tissue permeation enhancers, solubilizers, and adjuvants. Other therapeutic agents may be included, e.g., vasoconstrictors, anti-inflammatory agents, antibiotics, and counter-irritants. The compounds may be formulated using conventional techniques such as those described in Remington's Pharmaceutical Sciences, Mace Publishing Co., Philadelphia, Pa. 17^{th} Ed. (1985) and “Modern Pharmaceutics,” Marcel Dekker, Inc. 3^{rd} Ed. (G. S. Banker & C. T. Rhodes, Eds.). Pharmaceutically acceptable salts of the active agents (e.g., acid addition salts) may be prepared using standard procedures known to those skilled in the art of synthetic organic chemistry and described, e.g., by J. March, Advanced Organic Chemistry: Reactions, Mechanisms and Structure, 4^{th} Ed. (New York: Wiley-Interscience, 1992).

Most local anesthetics produce some degree of vasodilation, and they may be rapidly absorbed after local injection. A vasoconstrictor may therefore be added to enhance potency, reduce the maximum systemic concentration, and prolong the duration of action by localizing the active anesthetic molecule in local tissues. Adrenaline (epinephrine) and phenylepinephrine are commonly used vasoconstrictors, and may be added in concentrations ranging from about 1 in 80,000 to about 1 in 500,000, usually at about 1 in 200,000 (5 μg/ml) concentration. In particular, the duration of action is prolonged by the addition of adrenaline when used for infiltration anesthesia and peripheral nerve blocks. Adrenaline may also increase the duration of extradural anesthesia.

Corticosteroids that are useful to prolong in vivo nerve blockade include glucocorticoids such as dexamethasone, cortisone, hydrocortisone, prednisone, and others routinely administered orally or by injection. Other glucocorticoids include beclomethasone, betamethasone, flunisolide, methyl prednisone, para methasone, prednisolone, triamcinolome, alclometasone, amcinonide, clobetasol, fludrocortisone, diflurosone diacetate, fluocinolone acetonide, fluoromethalone, flurandrenolide, halcinonide, medrysone, and mometasone, and pharmaceutically acceptable salts and mixtures thereof.

Permeability enhancers may also be included. Permeability enhancers are used to aid in the passage of an ester anesthetic into a tissue or across a cell membrane. Typical enhancers may include bile salts such as sodium cholate, sodium glycocholate, sodium glycodeoxycholate, taurodeoxycholate, sodium deoxycholate, sodium lithocholate chenocholate, chenodeoxycholate, ursocholate, ursodeoxycholate, hydrodeoxycholate, dehydrocholate, glycochenocholate, taurochenocholate, and taurochenodeoxycholate. Other permeation enhancers such as urea, sodium dodecyl sulfate (SDS), dimethyl sulfoxide (DMSO), sodium lauryl sulfate, salts and other derivatives of saturated and unsaturated fatty acids, surfactants, bile salt analogs, derivatives of bile salts, or such synthetic permeation enhancers as described in U.S. Pat. No. 4,746,508 may also be used.

Lipophilic and/or amphiphilic solvents can be added to the carrier to prolong nerve blockade local anesthesia. These materials are well known to those skilled in the art and available from a variety of commercial sources. Examples of solvents include alcohols such as ethanol added in a dosage equivalent to approximately 1% alcohol, polyoxyethylene sorbitan derivatives such as polysorbate-80 or Tween, added in a concentration equivalent to between 1% and 3%.

For topical use, the compositions of the present invention can be in the form of emulsions, creams, jelly, solutions, and ointments containing, for example, up to about 10% by weight, preferably up to about 5% by weight of the combination of ester anesthetics. For parenteral administration, the compositions can be in the form of sterile injectable solutions and sterile packaged powders. Preferably, injectable solutions are formulated at a pH of about 4.5 to about 7.5. Some examples of suitable excipients include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, and methylcellulose. The formulations can additionally include lubricating agents such as talc, magnesium stearate, and mineral oil; wetting agents, emulsifying and suspending agents; preserving agents such as methyl- and propylhydroxy-benzoates, sweetening agents, and flavoring agents. The compositions may also comprise biodegradable polymer beads and dextran and cyclodextrin inclusion complexes.

V. Methods of Administration

The combination of ester anesthetics may be administered as a single injection or continuously through an indwelling catheter, or administered topically to the skin, mucus membranes, etc. The solution containing the combination of ester anesthetics may be administered repeatedly, e.g., at least 2, 3, 4, 5, 6, 7, 8, or more times, or the ester anesthetic that provides a long duration of effect may be administered separately in repeated doses, e.g., at least 2, 3, 4, 5, 6, 7, 8, or more times, after the administration of the ester anesthetic combination, or the solution may be administered by continuous infusion.

The compositions of the present invention may be administered by any of the accepted modes of administration of agents having similar utilities, for example, by topical, mucosal, or by parenteral routes, e.g., intradermal, intravenous, subcutaneous, intramuscular, epidural, rectal, vaginal, etc. The most suitable route will depend on the nature and severity of the condition being treated. Infiltration methods, which include peripheral nerve blocks, serosal, and neuraxial delivery, e.g. epidural, caudal, etc., are preferred routes for the compounds of this invention. Other suitable methods of administration include, but are not limited to, the placement of pledgets coated with a combination of ester anesthetics, intratracheal ester anesthetic administration, and ester anesthetic administration in the balloon of an endotracheal tube.

The compositions of the present invention may be administered for the production of analgesia and/or anesthesia and are particularly useful for the prophylaxis and/or treatment of pain. As local anesthetics, the compositions are useful for regional anesthesia, e.g., topical anesthesia, infiltration anesthesia, perisurgical tissue infiltration anesthesia, field block anesthesia, peripheral nerve block anesthesia, epidural anesthesia, spinal anesthesia, bier block anesthesia, and combinations thereof. In addition, the compositions may be administered for the relief of pain associated with venipuncture, lumbar puncture, myringotomy, and arterial cannulation. The compositions have other therapeutic applications including treatment and/or prophylaxis of neuropathic pain, trauma, and tissue ischemia.

As used herein, the term “topical anesthesia” refers to the administration of the compositions of the present invention to mucous membranes, the upper and lower respiratory tract, the skin, and the like. Examples of mucous membranes include, but are not limited to, the nose, the mouth, the esophagus, the tracheobronchial tree, and the genitourinary tract. Topical anesthetic formulations of the present invention are useful in the relief of pain due to dermatoses, hemorrhoids, and minor burns, as well as in the reduction of pain associated with sigmoidoscopy and in upper airway anesthesia prior to direct laryngoscopy. Topical anesthesia of the skin includes administration by patches or other reservoir systems, by bandages or gauzes containing local anesthetic, and by creams, ointments, sprays, or other transdermal drug administration systems known to one of skill in the art.

As used herein, the term “infiltration anesthesia” refers to the administration of the compositions of the present invention by extravascular placement in the region to be anesthetized. Preferably, infiltration anesthetic formulations of the present invention are administered by injection. The injection is in an immediate area of a surgical or painful stimulus, or in the tissues through which nerves transmitting painful signals pass. As used herein, “peripheral nerve block anesthesia” refers to the administration of the present invention by extravascular placement in the tissues immediately adjacent to major nerves, such as the nerves of the upper extremity (e.g., interscalene block, supraclavicular block, infraclavicular block, axillary block, median nerve block, radial nerve block, musculocutaneous nerve block, ulnar nerve block, digit block), the nerves of the lower extremity (e.g., lumbar plexus block, sciatic nerve block, femoral nerve block, obturator nerve block, lateral femoral cutaneous nerve block, peroneal nerve block, tibial block, ankle block), as well as block of major plexuses (e.g., cervical plexus, brachial plexus, celiac plexus, sacral plexus, femoral plexus). As used herein, the term “epidural anesthesia” refers to anesthesia caused by local anesthetic solutions injected into the epidural or sacral caudal space. The term “spinal anesthesia” refers to anesthesia following a local anesthetic injection into the lumbar subarachnoid space. Preferably, spinal anesthesia is performed with a formulation containing a combination of 2-chloroprocaine and tetracaine. The term “bier block anesthesia” refers to a local anesthetic injection into an extremity isolated by a tourniquet.

In contrast to amide-based local anesthetics, ester anesthetics are very rapidly metabolized and inactivated in the blood and many body tissues. Ester anesthetics therefore have a much lower potential for toxicity because their systemic concentrations are very small, except when administered by direct intravenous injection. As a result of the very low systemic concentrations, ester anesthetics offer the potential for at least two different ester anesthetics to be combined at their full respective doses to provide improved local anesthetic and/or analgesic activity. Such combinations are not possible with amide anesthetics because their far slower metabolism leads to high concentrations of the anesthetic in the tissue and blood. Since the toxicity of all local anesthetics is additive, the high blood and tissue concentrations that would result from a combination of full doses of two amide anesthetics would likely expose the patient to a significant risk of systemic toxicity, including seizures, cardiovascular collapse, and death. Further, because the problem of toxicity has been overcome with the compositions of the present invention, an optimal, analgesically and/or anesthetically effective amount of each ester anesthetic can be administered to a subject in need thereof. By contrast, when combinations of amide anesthetics are administered, it is necessary to use sub-optimal doses of each amide anesthetic because of the expected high blood levels of each amide anesthetic and the documented additive toxicity of local anesthetics. For example, the anesthetically effective amounts of lidocaine (2%) and bupivicaine (0.5%), if used in combination, would have very high toxicity and could potentially cause death. See, for example, Mets et al., Anesth. Analg., 75:611-614 (1992); Naguib et al., Drug Saf., 18:221-250 (1998); Kytta et al., Reg. Anesth. 16:89-94 (1991).

Currently used local anesthetics such as lidocaine produce pain and discomfort (e.g., stinging) when administered to a subject. By contrast, the compositions of the present invention, e.g., a combination of 2-chloroprocaine and tetracaine, produce a much lower level of pain and discomfort. Therefore, such compositions are not only easier to administer, but are also are better tolerated by a subject upon administration.

The present invention provides at least one ester anesthetic that has a rapid onset of action, where the onset of anesthetic effect begins within at least about 10 minutes of anesthetic administration, e.g., when delivered for infiltration anesthesia, and may be at least about 5 minutes, preferably at least about 2 minutes, or less. Suitable anesthetics for this purpose include procaine and 2-chloroprocaine. Preferably, the anesthetic is 2-chloroprocaine.

The present invention also provides at least one ester anesthetic that has an extended period of action, where an anesthetic effect from a single dose, for example, when delivered by infiltration anesthesia, is maintained for at least about 1 hour, preferably at least about 2 hours, more preferably at least 4 hours, still more preferably at least about 6 hours, or more.

As with all local anesthetics, the dose administered will vary depending on the anesthetic procedure, the vascularity of the tissues, the depth of anesthesia, the degree of muscle relaxation required, the duration of anesthesia desired, and the physical condition of the patient. Preferably, the smallest dose and concentration required to produce the desired result should be used. Dosage should be appropriately adjusted for children, the elderly, debilitated patients, and patients with cardiac and/or liver disease.

The following guidelines are not intended to imply any limitation to the dose that may be used in the methods and compositions of the present invention, but may indicate some guidelines for formulation. The following guidelines generally refer to a maximum single dose, where the amount may be increased by fractionation of the dose. As previously described, the rapid inactivation of ester-based anesthetics after administration provides a wide margin of safety for repeated administration of the anesthetic combination.

For example, the dose of 2-chloroprocaine given as a single injection in adults may be, without epinephrine, about 10, about 25, about 50, to about 100 mg/kg. Although clinical guidelines have suggested a maximum total dose of 800 mg, more recent data suggest a much higher dose can be administered without toxic effects, e.g., a maximum of about 1500 mg, of about 2500 mg, of about 5000 mg, usually not more than about 10,000 mg. The addition of epinephrine, e.g., at 1:200,000 concentration, allows an increase of about 20% in the dose of anesthetic. Earlier guidelines have suggested a maximum single dose of tetracaine to about 20 mg for spinal anesthesia. However, such doses may be exceeded, to a maximum of about 50 mg, of about 200 mg, usually not to exceed about 400 mg. The maximum single dose of procaine is about 1000 mg. For higher doses, care must be taken not to inject the anesthetic directly into a blood vessel.

The compositions are preferably formulated in a unit dosage form. The term “unit dosage forms” refers to physically discrete units suitable as unitary dosages for human subjects and other mammals, each unit containing a predetermined quantity of active material calculated to produce the desired onset, tolerability, and therapeutic effects, in association with a suitable pharmaceutical excipient (e.g., an ampoule). In addition, more concentrated compositions may be prepared, from which the more dilute unit dosage compositions may then be produced. The more concentrated compositions thus will contain substantially more than an anesthetically or analgesically effective amount of the compounds of the present invention, e.g., at least 1, 2, 3, 4, 5, 6, 7, 8, 9, 10, or more times the amount.

The compounds of the present invention, or their pharmaceutically acceptable salts, are administered in an analgesically and/or anesthetically effective amount. The duration of action and/or potency of the local anesthetic drug effect will be increased by comparison with the local anesthetic effect of formulations containing only a single ester-based local anesthetic, and will produce a duration of anesthesia equal to or greater than that produced by more toxic formulations of amide-based local anesthetics (see, Examples 2 and 3). Therefore, the dosage and dosing schedule may be adjusted accordingly. It will be understood that although the ratio of the two ester-based local anesthetics in the formulation will be fixed, the total volume of the formulation actually administered will be determined by a physician, in the light of the relevant circumstances, including the condition to be treated, the chosen route of administration, the actual compound administered and its relative activity, the age, weight, and response of the individual patient, the severity of the patient's symptoms, and the like.

The anesthetic combination of the invention can be tested for activity in various well-known assays (e.g., the batrachotoxin (BTX) displacement assay (McNeal et al., J. Med. Chem. 28: 381 (1985)), patch clamp method (see, Neher and Sakmann, “The Patch Clamp Technique “Scientific American” pp. 44-51 (1992); Hamill et al., Pflugera Arch. 391:85 (1981); intact isolated nerve assay, e.g., isolated frog sciatic nerve; blockage of the cutaneous trunci muscle reflex (CTMR) in guinea pigs (Bulbring et al., J. Pharmacol. Exp. Therap. 85:78-84 (1945); Blight et al., J. Compar. Neurology 296:614-633 (1990; Choi et al., Life Sci. 61: PL177-84 (1997)). Evaluation of motor and sympathetic function during sciatic nerve block in the rat is described, e.g., in Grant et al., Anesth. Analg. 75:889-94 (1992), and Thalhammer et al., Anesthesiology 82:1013-25 (1995).

### VI. EXAMPLES

The following examples are offered to illustrate, but not to limit, the claimed invention.

### Example 1

### Formulations

Solution for Injection A

| Ingredient | Quantity |
|---|---|
| Sodium Chloride | 0.9% (0.9 g/100 mL) |
| Epinephrine | 1 μg/ml |
| NaBicarbonate | As necessary to prevent acid catalyzed ester |
| | hydrolysis in vitro; not to exceed quantities |
| | capable of inducing precipitation of the free |
| | base form of the local anesthetic. |
| 2-chloroprocaine | 10 mg/ml |
| Tetracaine | 1 mg/ml |
| Water for injection | to 100 ml |

Solution for Injection B

| Ingredient | Quantity |
|---|---|
| Sodium Chloride | 0.9% (0.9 g/100 mL) |
| Epinephrine | 2 μg/ml |
| NaBicarbonate | As necessary to prevent acid catalyzed ester |
| | hydrolysis in vitro: not to exceed quantities |
| | capable of inducing precipitation of the free |
| | base form of the ocal anesthetic. |
| 2-chloroprocaine | 30 mg/ml |
| Tetracaine | 3 mg/ml |
| Water for injection | to 100 ml |

Solution for Injection C

| Ingredient | Quantity |
|---|---|
| Sodium Chloride | 0.9% (0.9 g/100 mL) |
| Epinephrine | 5 μg/ml |
| NaBicarbonate | As necessary to prevent acid catalyzed ester |
| | hydrolysis in vitro; not to exceed quantities |
| | capable of inducing precipitation of the free |
| | base form of the local anesthetic. |
| 2-chloroprocaine | 100 mg/ml |
| tetracaine | 10 mg/ml |
| Water for injection | to 100 ml |

Paste

| Ingredient | Quantity % | |
|---|---|---|
| Zinc oxide | 25 | |
| Starch | 25 | |
| Calamine | 5 | |
| 2-chloroprocaine | 30 | mg/ml |
| tetracaine | 5 | mg/ml |
| White petrolatum | to 100 | ml |

Ointment

| Ingredient | Quantity % | |
|---|---|---|
| 2-chloroprocaine | 30 | mg/ml |
| tetracaine | 5 | mg/ml |
| White wax | 5 | |
| White petrolatum | to 100 | ml |

Cream

| | |
|---|---|
| Ingredient | Quantity % |

Oleaginous phase

| | |
|---|---|
| 2-chloroprocaine | 30 mg/ml |
| tetracaine | 5 mg/ml |
| Spermaceti | 12.5 |
| White wax | 12.0 |
| Almond oil | 55.5 |

Aqueous phase

| | |
|---|---|
| Sodium borate | 0.5 |
| Stronger rose water | 2.5 |
| Purified water | 16.5 |
| Aromatic Rose oil | 0.02 |

Gel

| Ingredient | Quantity % |
|---|---|
| 2-chloroprocaine | 30 mg/ml |
| tetracaine | 5 mg/ml |
| Methocel 90 H.C. 4000 | 0.8 |
| Carbopol 934 | 0.24 |
| Propylene glycol | 16.7 |
| Methylparaben | 0.015 |
| Purified water | to 100 |

### Example 2

### Comparison of Local Anesthetics Administered Individually or in Combination

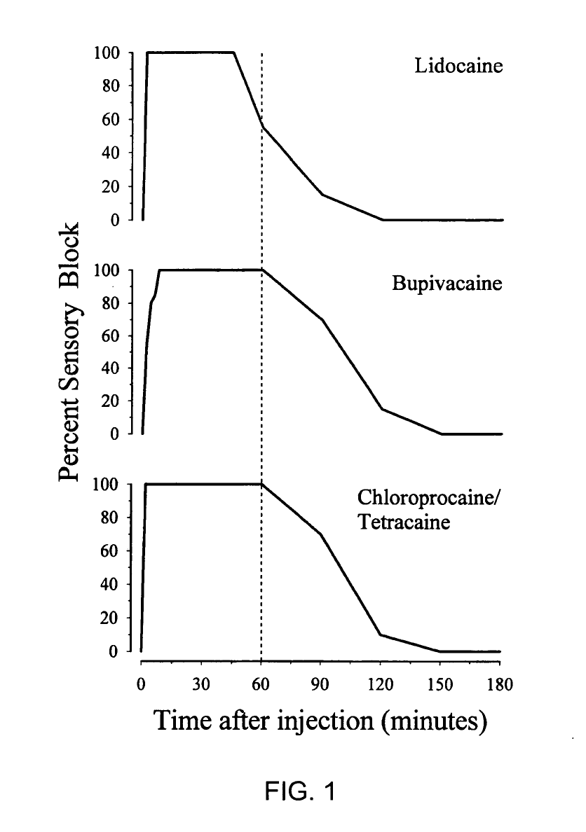

The onset and offset of sciatic nerve block from lidocaine, bupivacaine, tetracaine, and 2-chloroprocaine were examined in studies conducted in rats. Three different modalities were tested: (1) pain (e.g., withdrawal to pinch); (2) proprioception (e.g., the ability to place the paw squarely on the table); and (3) motor strength (e.g., the downward pressure the paw can exert on a scale). In each case, the same volume of local anesthetic agent was tested. The modality of interest was withdrawal to pinch. Each local anesthetic was tested on 5 rats of nearly identical size and age. The results are shown in FIG. 1.

The upper panel in FIG. 1 shows the time course of sciatic nerve block with 2.0% lidocaine, the strongest concentration of lidocaine commercially available. At this concentration, lidocaine produced a nearly immediate onset of block (i.e., 100% at the first time point tested, occurring 2 minutes after injection). However, the duration of the sensory block was about 45 minutes, at which point it started to decay.

The middle panel in FIG. 1 shows the time course of sciatic nerve block with an identical volume of 0.5% bupivacaine injected next to the rat sciatic nerve. The onset of sensory block was 4 times slower than with 2.0% lidocaine, requiring 8 minutes to achieve full sensory block in all animals tested. However, a good sensory block persisted until 60 minutes, at which point it started to decay.

The lower panel in FIG. 1 shows the time course of sciatic nerve block from administering a combination of tetracaine and 2-chloroprocaine. The formulation contained 0.17% tetracaine and 2.3% 2-chloroprocaine. This experiment demonstrates that the administration of a combination of local anesthetics provides an onset of sensory block that was as rapid as with lidocaine (e.g., with full onset by about 2 minutes) and a duration of sustained activity (e.g., complete sensory block) that was substantially longer than with lidocaine alone. In fact, the duration of complete sensory block exceeded that observed with bupivacaine. As such, administration in a single injection of a combination of local ester anesthetics such as tetracaine and 2 chloroprocaine can provide an onset of sensory block comparable to lidocaine and a duration of sustained sensory block equivalent to or superior to bupivacaine.

### Example 3

### Demonstration of Synergy of Local Anesthetics Administered in Combination

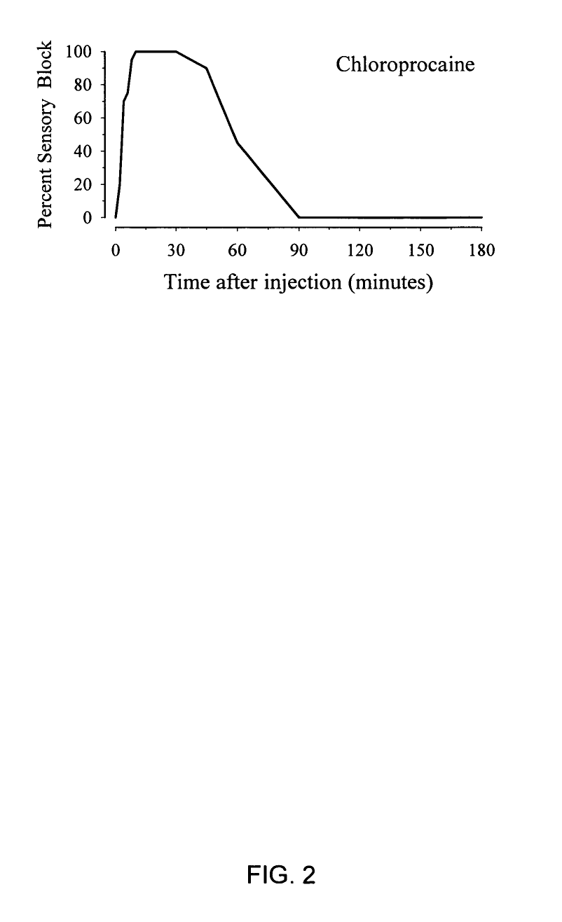

FIG. 2 shows the onset and duration of a rat sciatic nerve block after administration of 2% 2-chloroprocaine. The duration of the block was substantially shorter without co-administration of tetracaine, with full sensory blockade lasting only about 30 minutes. In addition, the onset of the block with 2-chloroprocaine administration was slower than with co-administration of tetracaine, suggesting that tetracaine acts synergistically to enhance the onset of 2-chloroprocaine anesthesia.

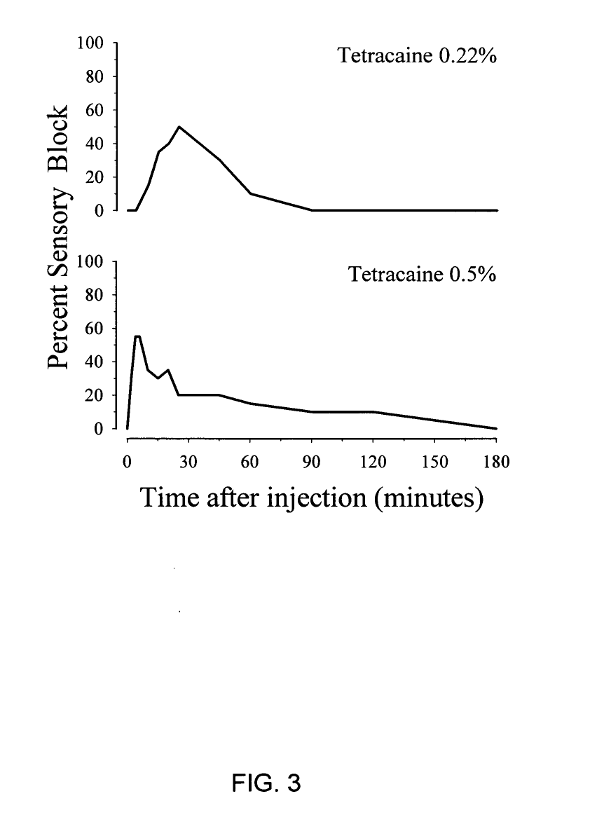

FIG. 3 shows the onset and duration of a sciatic nerve block after administration of either 0.22% tetracaine (upper panel) or 0.5% tetracaine (lower panel). Strikingly, both concentrations of tetracaine failed to produce an adequate sensory block (e.g., less than 60% sensory block). By contrast, complete sensory block was achieved when tetracaine, at a lower concentration, was co-administered with 2-chloroprocaine (see, FIG. 1, lower panel). Further, the partial sensory block produced by the administration of either 0.22% tetracaine or 0.5% tetracaine displayed not only a slow onset, but also dissipated rapidly. Multiple studies using different doses of tetracaine confirmed these results, as not a single rat demonstrated profound and sustained sensory blockade with any concentration of tetracaine teasted. All animals used were evaluated for correct placement of the local anesthetic injection in proximity to the sciatic nerve by demonstrating complete, prolonged motor blockade (data not shown). Thus, these results show that tetracaine by itself is a poor local anesthetic for producing sensory blockade of the rat sciatic nerve with rapid onset and a long duration of effect.

Further, these results demonstrate that the rapid onset and long duration of effect achieved upon administration of a combination of tetracaine and 2-chloroprocaine is clearly more than the sum of the independent effects of tetracaine and 2-chloroprocaine alone. More particularly, in the absence of 2-chloroprocaine, tetracaine does not provide an adequate sensory block at any point in time. Similarly, in the absence of tetracaine, 2-chloroprocaine produces a sensory block that is very transient. Only the combination of the two local anesthetics is able to produce a sensory block that lasts for more than 60 minutes. As such, tetracaine and 2-chloroprocaine, when administered in combination, act synergistically to provide safe, complete, rapid, and long-lasting local anesthetic activity.

### Example 4

### Determination of Neurotoxicity Following Local Anesthetic Administration

Sections of rat peroneal nerve were taken for evaluation with light microscopy five days post injection with local anesthetic(s). Specifically, each nerve was sectioned and total axonal counts were taken to assess any axonal loss. Five nerves were examined for each treatment condition. The peroneal axonal counts five days after control (saline), bupivacaine, and 2.3% 2-chloroprocaine/0.17% tetracaine injections were 2063±30 (SEM), 1959±81, and 1993±45, respectively. There was no significant difference in total axonal counts between bupivacaine, 2-chloroprocaine/tetracaine, and saline controls. In contrast, the total axonal counts for nerves treated with 2.0% lidocaine were 1602±167, which was significantly reduced from the saline controls, demonstrating significant neurotoxicity following lidocaine administration.

Sections were also taken across the sciatic nerve five days after injection with local anesthetic(s) and examined for gross pathological injury. Light microscopic sections of the sciatic nerve at the approximate site of the injection of 2.3% 2-chloroprocaine/0.17% tetracaine and 0.5% bupivacaine were indistinguishable from saline controls, while the sciatic nerves at the site of the 2.0% lidocaine injections demonstrated marked edema and neural damage, indicating significant neurotoxicity following lidocaine administration.

These experiments demonstrate that the combination of 2-chloroprocaine/tetracaine is not neurotoxic and is indistinguishable histopathologically from saline controls. By contrast, 2.0% lidocaine demonstrated significant neurotoxicity both at the level of the peri-sciatic injection and in the distal peroneal nerve.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A method for local anesthesia, the method comprising:
administering a combination of at least two ester anesthetics.

2. The method according to claim 1, wherein at least one of said ester anesthetics provides a rapid onset of action, and at least one of said ester anesthetics provides a long duration of effect.

3. The method according to claim 2, wherein one of said ester anesthetics is procaine or 2-chloroprocaine.

4. The method according to claim 2, wherein one of said ester anesthetics is tetracaine.

5. The method according to claim 2, wherein one of said ester anesthetics is 2-chloroprocaine and one is tetracaine.

6. The method according to claim 2, wherein said combination of at least two ester anesthetics is administered as a single pharmaceutical formulation.

7. The method according to claim 6, wherein said pharmaceutical formulation further comprises a buffering agent.

8. The method according to claim 6, wherein said formulation further comprises a vasoconstrictive agent.

9. The method according to claim 8, wherein said vasoconstrictive agent is epinephrine or phenylepinephrine.

10. The method according to claim 6, wherein said formulation further comprises a corticosteroid.

11. The method according to claim 6, wherein said formulation further comprises a tissue permeability enhancer.

12. The method according to claim 2, wherein said combination of ester anesthetics is delivered topically.

13. The method according to claim 2, wherein said combination of ester anesthetics is delivered by infiltration.

14. The method according to claim 13, wherein said infiltration achieves a peripheral nerve block.

15. The method according to claim 13, wherein said infiltration achieves an epidural nerve block.

16. The method according to claim 13, wherein said infiltration achieves a caudal nerve block.

17. A pharmaceutical composition for local anesthesia, said pharmaceutical composition comprising:
(a) a combination of at least two ester anesthetics; and
(b) a pharmaceutically acceptable carrier.

18. The pharmaceutical composition according to claim 17, wherein at least one of said ester anesthetics provides a rapid onset of action, and at least one of said ester anesthetics provides a long duration of effect.

19. The pharmaceutical composition according to claim 18, wherein one of said ester anesthetics is procaine or 2-chloroprocaine.

20. The pharmaceutical composition according to claim 18, wherein one of said ester anesthetics is tetracaine.

21. The pharmaceutical composition according to claim 18, wherein one of said ester anesthetics is 2-chloroprocaine and one is tetracaine.

22. The pharmaceutical composition according to claim 21, wherein said 2-chloroprocaine is present at a concentration of from about 1% to about 3%.

23. The pharmaceutical composition according to claim 21, wherein said tetracaine is present at a concentration of from about 0.1% to about 0.5%.

24. The pharmaceutical composition according to claim 17, wherein said pharmaceutical formulation further comprises a buffering agent.

25. The pharmaceutical composition according to claim 17, wherein said formulation further comprises a vasoconstrictive agent.

26. The pharmaceutical composition according to claim 25, wherein said vasoconstrictive agent is epinephrine or phenylepinephrine.

27. The pharmaceutical composition according to claim 17, wherein said formulation further comprises a corticosteroid.

28. The pharmaceutical composition according to claim 17, wherein said formulation further comprises a tissue permeability enhancer.
